(19) United States
(12) Patent Application Publication
(10) Pub. No.: US 2026/0193301 A1
(43) Pub. Date: Jul. 9, 2026

(54) Application of NPE1 Protein and Biomaterial Encoding NPE1 Protein in Regulating Catalytic Efficiency of Metabolic Enzyme

(71) Applicants: Sanya Institute of Henan University, Sanya City (CN); Henan University, Zhengzhou City (CN)

(72) Inventors: Xuelu WANG, Sanya (CN); Xiaolong KE, Sanya (CN); Han XIAO, Sanya (CN)

(21) Appl. No.: 19/432,046

(22) Filed: Dec. 23, 2025

(30) Foreign Application Priority Data
Jan. 8, 2025 (CN) 202510025298X

(51) Int. Cl. C07K 14/415; A61K 38/00

(52) U.S. Cl. CPC C07K 14/415; A61K 38/00

(57) ABSTRACT

The present invention identifies and provides an NPE1 protein and a biomaterial encoding the NPE1 protein, which are applicable in regulating the catalytic efficiency of metabolic enzymes. The new soybean gene, NPE1, in soybeans, can regulate the catalytic efficiency of key metabolic enzymes. Overexpression of NPE1 in soybeans will significantly enhance the catalytic efficiency of key metabolic enzymes GmPGK3a, GmPFK3a, GmGS1γ1, and GmPGD2a in nodules, thereby enhancing the nitrogen fixation ability of soybean nodules and increasing the aboveground biomass and ultimate yield of the plant. The present invention provides genetic resources for nitrogen-efficient and molecular breeding of legumes including soybeans, and provides a theoretical basis for the molecular design of high-efficiency nitrogen-fixing soybean varieties, thereby promoting the development of environmentally friendly green and sustainable agriculture.

### CROSS REFERENCE OF RELATED APPLICATION

This is a non-provisional application that claims priority to Chinese application number 202510025298X, filing date Jan. 8, 2025, the entire contents of each of which are expressly incorporated herein by reference.

### BACKGROUND OF THE PRESENT INVENTION

### Field of Invention

The present invention belongs to the technology field of biotechnology, and is related to a new application of NPEI protein.

### Description of Related Arts

As a traditionally agricultural nation, our country (China) has achieved production of staple grains such as rice and wheat exceeding 650 million tons for eight consecutive years as of 2022. At the current stage, the production of staple food in our country can basically meet the food needs of our residents. As living standards improve, demand for nutritious foods like meat, eggs, and milk is growing, making us a major feed consumer, of which soybean feed is a major component. Furthermore, soybean oil accounts for a staggering 58.4% of edible oil consumption and is a major component of edible oil consumption in our country. Therefore, the annual demand for soybeans in our country is enormous. However, domestic soybean production currently only meets domestic consumption, while soybean crushing, which accounts for 85% of total soybean consumption, relies on imports. In 2020, my country's soybean imports exceeded 100 million tons. Long-term dependence on soybean imports is not conducive to national food security and the stability of the soybean industry structure. Therefore, developing soybean production and achieving “soybean revitalization” in our country are of great significance to ensuring national food security.

Nitrogen is a macronutrient essential for plant growth and development and a key component of molecules such as proteins and nucleic acids in living organisms. Although 78.1% of Earth's atmosphere contains nitrogen, most plants cannot directly utilize it. Therefore, agricultural production is highly dependent on industrial nitrogen fertilizers. However, the production of nitrogen fertilizer requires large amounts of fossil fuels, and excessive nitrogen fertilizer application can cause soil compaction and degradation, as well as water pollution, impacting sustainable agricultural development. Biological nitrogen fixation is the largest natural source of bioavailable nitrogen in nature. Legumes and rhizobia interact to form a unique organ called the symbiotic nodule. In global ecosystems, symbiotic nitrogen fixation between legumes and rhizobia accounts for 60%-70% of total biological nitrogen fixation. Symbiotic nitrogen fixation between legumes and rhizobia not only reduces energy consumption but also contributes to soil improvement, making it an important way to improve the nitrogen efficiency of legume crops. In agricultural systems, soybean nitrogen fixation accounts for more than 86% of the total symbiotic nitrogen fixation of legumes, with an annual nitrogen production of 16.44 million tons. Therefore, improving the efficiency of symbiotic nitrogen fixation between soybeans and rhizobia is of great significance to the development of green and sustainable agriculture

Symbiotic nitrogen fixation in root nodules is an energy-intensive biological process. Carbohydrates produced by photosynthesis in legumes are the primary energy source for symbiotic nitrogen fixation. The photosynthetic product, in the form of sucrose, is transported through the phloem to mature nodules. After the photosynthetic product reaches the nodules, sucrose is broken down into hexose by sucrose synthase and alkali invertase and then enters the glycolysis pathway to produce phosphoenolpyruvate (PEP).

PEP can generate malic acid through a two-step reaction catalyzed by phosphoenolpyruvate carboxylase (PEPC) and malate dehydrogenase (MDH). Malic acid is transported into the bacteroid to undergo the tricarboxylic acid cycle to produce ATP to provide energy for the nitrogen fixation reaction. The ammonium ions generated by nitrogen fixation are then assimilated into asparagine and glutamine in the amide-exporting legumes and transported to the aboveground parts. In ureide-exporting legumes, which is generally represented by soybeans, glutamine generated by ammonium radicals enters the de novo purine biosynthesis pathway to produce inosine monophosphate (IMP), which is ultimately converted into ureides and transported to the aboveground parts. Also, PEP can generate pyruvate, the final product of glycolysis, under the catalysis of pyruvate kinase (PK). Pyruvate then enters the mitochondrial tricarboxylic acid cycle to produce ATP to provide energy for nitrogen assimilation in nodules.

While the carbon and nitrogen metabolic pathways in soybean nodules are relatively well understood, the genes and regulatory mechanisms that regulate the activities of key catalytic enzymes in these pathways remain largely unresolved. The primary technical challenges our research team aims to address are how to regulate the catalytic efficiency of key metabolic enzymes in soybean nodules and how to leverage this regulatory mechanism to enhance soybean's symbiotic nitrogen fixation capacity, thereby improving soybean nitrogen utilization. In addition, since these metabolic pathways and key metabolic enzymes are highly conserved in different species, whether this regulatory mechanism can be applied to humans and other species to solve metabolic-related diseases is also a technical issue we want to explore.

### SUMMARY OF THE PRESENT INVENTION

In order to solve the above technical problems, the present invention provides an NPE1 protein and a biomaterial encoding the NPE1 protein for application in regulating the catalytic efficiency of metabolic enzyme.

The technical solution of the present invention is realized as follows:

A protein that controls the catalytic efficiency of metabolic enzymes. The protein is a small peptide consisting of 26 amino acids.

The above proteins can interact with phosphoglycerate kinase GmPGK3a, phosphofructokinase GmPFK3a, glutamine synthetase GmGS1γ1 and 6-phosphogluconate dehydrogenase GmPGD2a in soybean and improve their catalytic efficiency, thereby enhancing the nitrogen fixation ability of soybean nodule.

Furthermore, the protein can also enhance the catalytic ability of human phosphoglycerate kinase HsPGK1.

Furthermore, the protein can also regulate the catalytic ability of human phosphofructokinase HsPFKP.

The protein that controls the catalytic efficiency of metabolic enzymes is NPE1 protein, and its amino acid sequence is shown in SEQ ID No.3.

The nucleotide sequence of the NPE1 gene encoding the NPE1 protein is shown in SEQ ID No. 1, and the nucleotide sequence of the coding region of the NPE1 gene after removing introns is shown in SEQ ID No. 2.

An overexpression vector containing the aforesaid NPE1 gene.

An application of the aforesaid protein or gene in improving plant symbiotic nitrogen fixation efficiency, enhancing plant environmental adaptability and increasing plant yield.

An application of the aforesaid protein or gene in medicine and pharmaceuticals.

An application of the aforesaid overexpression vector in improving plant symbiotic nitrogen fixation efficiency, enhancing plant environmental adaptability and increasing plant yield.

An application of the aforesaid overexpression vector in medicine and pharmaceuticals.

Furthermore, the steps include: transferring the aforesaid overexpression vector into the plant to be improved through genetic transformation, then selecting transgenic positive plants for cultivation, and completing the application of enhancing the symbiotic nitrogen fixation ability of soybeans.

Preferably, the plant is a leguminous plant.

The present invention has the following advantageous effect:
1. The present invention successfully identifies a novel soybean gene, NPE1, and obtains its gene sequence through reverse genetics. The gene is successfully isolated, cloned, and verified using a soybean hairy root transient transformation system, stable transgenic plants, PCR reactions, and gene sequencing. The NPE1 gene encodes a small peptide NPE1 containing 26 amino acids. NPE1 can interact with phosphoglycerate kinase GmPGK3a, phosphofructokinase GmPFK3a, glutamine synthetase GmGS1γ1 and 6-phosphogluconate dehydrogenase GmPGD2a in soybean nodules and improve their catalytic efficiency.
2. According to the present invention, the soybean protein NPE1 can enhance the catalytic capacity of key metabolic enzymes GmPGK3a, GmPFK3a, GmGS1γ1 and GmPGD2a in nodules. By overexpressing the NPE1 gene in soybeans, the nitrogen fixation efficiency of soybean nodules can be significantly enhanced.
3. According to the present invention, overexpression of NPE1 in soybeans can significantly enhance the catalytic efficiency of key metabolic enzymes GmPGK3a, GmPFK3a, GmGS1γ1, and GmPGD2a in nodules, thereby enhancing the nitrogen fixation ability of soybean nodules and increasing the aboveground biomass and ultimate yield of the plant. The studies of the present invention will provide genetic resources for nitrogen-efficient and molecular breeding of legumes including soybeans, and provide a theoretical basis for the molecular design of high-efficiency nitrogen-fixing soybean varieties, thereby promoting the development of environmentally friendly, green and sustainable agriculture.
4. According to the present invention, the soybean protein NPE1 can also enhance the catalytic efficiency of human phosphoglycerate kinase HsPGK1. By using NPE1 protein in clinical practice, it may be possible to treat metabolic-related diseases.
5. According to the present invention, the soybean protein NPE1 can also can also regulate the catalytic efficiency of human phosphofructokinase HsPFKP. By using NPE1 protein in clinical practice, it may be possible to treat metabolic-related diseases.

### BRIEF DESCRIPTION OF THE DRAWINGS

In order to more clearly explain the embodiments of the present invention or the technical solutions in the prior arts, the drawings needed to be used in the description of the embodiments or the prior arts will be briefly introduced below. Obviously, the drawings in the following description are only some embodiments of the present invention. For those of ordinary skill in the art, based on these drawings without exerting creative efforts, other drawings can also be obtained.

FIG. 1 illustrates the interaction between NPE1 and GmPGK3a/GmPFK3a/GmGS1γ1/GmPGD2a in tobacco leaf epidermal cells.

FIG. 2 illustrates the in vitro interaction between NPE1 and GmPGK3a/GmPFK3a/GmGS1γ1/GmPGD2a.

FIG. 3 illustrates the effect of NPE1 and phosphorylated NPE1 protein on the catalytic efficiency of GmPGK3a.

FIG. 4 illustrates the effect of NPE1 and phosphorylated NPE1 protein on the catalytic efficiency of GmPFK3a.

FIG. 5 illustrates the effect of NPE1 and phosphorylated NPE1 protein on the catalytic efficiency of GmGS1γ1.

FIG. 6 illustrates the effect of NPE1 and phosphorylated NPE1 protein on the catalytic efficiency of GmPGD2a.

FIG. 7 illustrates the effect of overexpression of NPE1 and NPE1 with phosphorylation site mutation on the nitrogen fixation ability of soybean nodules.

FIG. 8 illustrates the effect of NPE1 and phosphorylated NPE1 protein on the catalytic efficiency of HsPGK1.

FIG. 9 illustrates the effect of NPE1 and phosphorylated NPE1 protein on the catalytic efficiency of HsPFKP.

### DETAILED DESCRIPTION OF THE PREFERRED EMBODIMENT

The technical solutions in the embodiments of the present invention will be clearly and completely described below with reference to the accompanying drawings in the embodiments of the present invention. Obviously, the embodiments described are only some of the embodiments of the present invention, rather than all the embodiments. Based on the embodiments of the present invention, all other embodiments obtained by those of ordinary skill in the art without exerting creative efforts fall within the scope of protection of the present invention.

Unless otherwise specified, the experimental methods used in the following experimental examples are all conventional methods; the materials and reagents used are commercially available reagents and materials unless otherwise specified.

### Embodiment 1

The present invention identified a new soybean gene NPE1 through the soybean genome and transcriptome databases published online, and the sequence is shown as SEQ ID No. 1.

The gene sequence of NPE1 is obtained by the following method:

The template is 1 μL (about 50 ng) of genomic DNA from soybean line W82, 5 μL of 10×KOD enzyme reaction buffer, 2 μL of 25 mM MgCl₂, 5 μL of 5 mM dNTP, 5 μL of 5 μM primers (NPE1-FP and NPE1-RP primers, 2.5 μL of each primer), and 1 μL of KOD enzyme, and add ddH₂O (sterile deionized water) to 50 μL.

The primers are:

NPE1-FP [SEQ. ID. NO. 26]:
ATGATGAAATGTGAAAATAT;
NPE1-RP [SEQ. ID. NO. 27]:
TCATGATGAAACTGCAACTG.

The reaction procedure is as follows:

Carry out denaturation for 35 cycles, each with denaturation at 94° C. for 5 minutes, at 94° C. for 30 seconds, 55° C. for 1 minute, and 68° C. for 2 minutes, and then carry out extension at 68° C. for 10 minutes.

Finally, a gene sequence containing the nucleotide sequence of SEQ ID No. 1 is obtained, and the protein encoding the gene is shown in SEQ ID No. 3.

### Embodiment 2

1. Using NPE1-YC-FP primer and NPE1-YC-RP primer, carry out amplification to obtain the NPE1 coding sequence (shown in SEQ ID No. 2); then insert the NPE1 coding sequence into the backbone vector pXY105 to obtain the fusion protein vector NPE1-YC expressing NPE1 and the carboxyl terminus of yellow fluorescent protein (YC). Using GmPGK3a-YN-FP primer and GmPGK3a-YN-RP primer, carry out amplification to obtain the GmPGK3a coding sequence; then insert the GmPGK3a coding sequence into the backbone vector pXY103 to obtain the fusion protein vector GmPGK3a-YN expressing GmPGK3a and the amino terminus of yellow fluorescent protein (YN). Using GmPFK3a-YN-FP primer and GmPFK3a-YN-RP primer, carry out amplification to obtain the GmPFK3a coding sequence; then insert the GmPFK3a coding sequence into the backbone vector pXY103 to obtain the fusion protein vector GmPFK3a-YN expressing GmPFK3a and the amino terminus of yellow fluorescent protein (YN). Using GmGS1γ1-YN-FP primer and GmGS1γ1-YN-RP primer, carry out amplification to obtain the GmGS1γ1 coding sequence; then insert the GmGS1γ1 coding sequence into the backbone vector pXY103 to obtain the fusion protein vector GmGS1γ1-YN expressing GmGS1γ1 and the amino terminus of yellow fluorescent protein (YN). Using GmPGD2a-YN-FP primer and GmPGD2a-YN-RP primer, carry out amplification to obtain the GmPGD2a coding sequence; then insert the GmPGD2a coding sequence into the backbone vector pXY103 to obtain the fusion protein vector GmPGD2a-YN expressing GmPGD2a and the amino terminus of yellow fluorescent protein (YN).

Wherein the ID of the GmPGK3a gene is Glyma.08G165400; the ID of the GmPFK3a gene is Glyma.07G126400; the ID of the GmGS1γ1 gene is Glyma. 14G213300; and the ID of the GmPGD2a gene is Glyma. 18G277300.

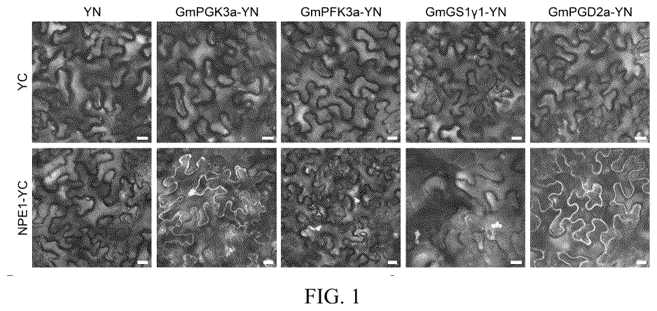

After YC-expressing pXY105, YN-expressing pXY103, and the aforesaid vectors are transformed into Agrobacterium tumefaciens GV3101, Agrobacterium tumefaciens GV3101 transformed with different vectors are mixed and then injected into Nicotiana tabacum leaves. Use Laser confocal microscopy to observe YFP fluorescence in leaf epidermal cells. It is found that after NPE1-YC is respectively mixed with GmPGK3a-YN, GmPFK3a-YN, GmGS1γ1-YN, and GmPGD2a-YN and injected, yellow fluorescence is observed, indicating that NPE1 can interact with GmPGK3a/GmPFK3a/GmGS1γ1/GmPGD2a in tobacco leaves (FIG. 1).

The primers are:

NPE1-YC-FP [SEQ. ID. NO. 4]:
TACTATTTACAATTACAGGTACCATGATGAAATGTGAAAATATAA
TCAAAC;
NPE1-YC-RP [SEQ. ID. NO. 5]:
ACGCTGCCCATACCGCCGTCGACTGATGAAACTGCAAAGTTCAT;
GmPGK3a-YN-FP [SEQ. ID. NO. 6]:
CAATTACAGGTACCCGGGGATCCATGGCGACTAAGAGGAGCGTG;
GmPGK3a-YN-RP [SEQ. ID. NO. 7]:
TTGCTCACCATACCGCCGTCGACAGCATCATCAAGGGCAAGGACA
C;
GmPFK3a-YN-FP [SEQ. ID. NO. 8]:
CAATTACAGGTACCCGGGGATCCATGGATTCTAATTCTTCTTCTC
CAAG;
GmPFK3a-YN-RP [SEQ. ID. NO. 9]:
TTGCTCACCATACCGCCGTCGACATGCTGGATTGTGTCACTTCCC
ATG;
GmGS1y1-YN-FP [SEQ. ID. NO. 10]:
CAATTACAGGTACCCGGGGATCCATGTCGTTGCTCTCCGATCTTA
TC;
GmGS1y1-YN-RP [SEQ. ID. NO. 11]:
TTGCTCACCATACCGCCGTCGACTGGTTTCCAAAGAATGGTTGTC
TC;
GmPGD2a-YN-FP [SEQ. ID. NO. 12]:
CAATTACAGGTACCCGGGGATCCATGGCTCAACCCACAACAAGAA
TAG;
GmPGD2a-YN-RP [SEQ. ID. NO. 13]:
TTGCTCACCATACCGCCGTCGACATTTCTTGACTGTTTGGCAAGC
TTG.

2. Using NPE1-GST-FP primer and NPE1-GST-RP primer, carry out amplification to obtain the NPE1 coding sequence (shown in SEQ ID No. 2); then insert the NPE1 coding sequence into the backbone vector pGEX4T1 to obtain NPE1-GST, the fusion protein vector expressing NPE1 and the glutathione S-transferase (GST). Using His-GmPGK3a-FP primer and His-GmPGK3a-RP primer, carry out amplification to obtain the GmPGK3a coding sequence; then insert the GmPGK3a coding sequence into the backbone vector pET28a to obtain His-GmPGK3a, the fusion protein vector expressing GmPGK3a and histidine (His). Using His-GmPFK3a-FP primer and His-GmPFK3a-RP primer, carry out amplification to obtain the GmPFK3a coding sequence; then insert the GmPFK3a coding sequence into the backbone vector pET28a to obtain His-GmPFK3a, the fusion protein vector expressing GmPFK3a and histidine (His). Using His-GmGS1γ1-FP primer and His-GmGS1γ1-RP primer, carry out amplification to obtain the GmGS1γ1 coding sequence; then insert the GmGS1γ1 coding sequence into the backbone vector pET28a to obtain His-GmGS1γ1 the fusion protein vector expressing GmGS1γ1 and histidine (His). Using His-GmPGD2a-FP primer and His-GmPGD2a-RP primer, carry out amplification to obtain the GmPGD2a coding sequence; then insert the GmPGD2a coding sequence into the backbone vector pET28a to obtain His-GmPGD2a, the fusion protein vector expressing GmPGD2a and histidine (His).

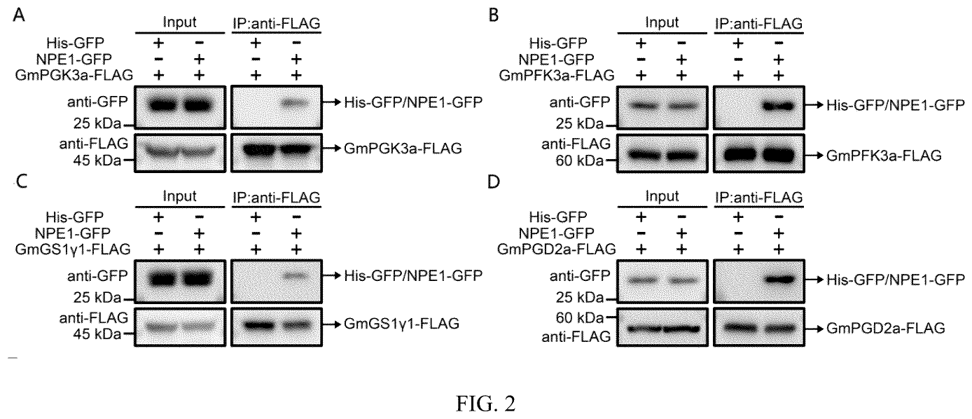

The aforesaid prokaryotic expression vectors are transformed into Escherichia coli BL21 strain. After shaking in LB medium, isopropyl-β-D-thiogalactopyranoside (IPTG) is added to having a final concentration of 0.8 mM and carrying out induction overnight at 16° C. to express NPE1-GST, His-GmPGK3a, His-GmPFK3a, His-GmGS1γ1, and His-GmPGD2a fusion proteins, and the proteins are purified using commercial agarose beads. Then, NPE1-GST is incubated with fusion proteins His-GmPGK3a, His-GmPFK3a, His-GmGS1γ1, and His-GmPGD2a, respectively. The expression of NPE1-GST is detected by Western blotting using anti-His agarose beads. After pulling down His-GmPGK3a/His-GmPFK3a/His-GmGS1γ1/His-GmPGD2a proteins, anti-GST antibody is used to detect whether NPE1-GST will interact with these proteins. The results show that NPE1-GST interacted with His-GmPGK3a/His-GmPFK3a/His-GmGS1γ1/His-GmPGD2a in the in vitro pull-down experiment (FIG. 2).

The primers are:

NPE1-GST-FP [SEQ. ID. NO. 14]:
CTTTAAGAAGGAGATATACTGCAGATGATGAAATGTGAAAATATA
ATCAAAC;
NPE1-GST-RP [SEQ. ID. NO. 15]:
GACATGCTACCACCACCACCGCTACCACCACCACCTGATGAAACT
GCAAAGTTCAT;
His-GmPGK3a-FP [SEQ. ID. NO. 16]:
GTGGACAGCAAATGGGTCGCGGATCCGCGACTAAGAGGAGCGTGG
GAAC;
His-GmPGK3a-RP [SEQ. ID. NO. 17]:
TCGAGTGCGGCCGCAAGCTTGTCGACTCAAGCATCATCAAGGGCA
AGGAC;
His-GmPFK3a-FP [SEQ. ID. NO. 18]:
GTGGACAGCAAATGGGTCGCGGATCCATGGATTCTAATTCTTCTT
CTCCAAG;
His-GmPFK3a-RP [SEQ. ID. NO. 19]:
TCGAGTGCGGCCGCAAGCTTGTCGACCTAGCAAGTIGTGGGATAA
ATGC;
His-GmGS1y1-FP [SEQ. ID. NO. 20]:
GTGGACAGCAAATGGGTCGCGGATCCTCGTTGCTCTCCGATCTTA
TCAAC;
His-GmGS1y1-RP [SEQ. ID. NO. 21]:
TCGAGTGCGGCCGCAAGCTTGTCGACTTATGGTTTCCAAAGAATG
GTTGTC;
His-GmPGD2a-FP [SEQ. ID. NO. 22]:
GTGGACAGCAAATGGGTCGCGGATCCATGGCTCAACCCACAACAA
GAATAG;
His-GmPGD2a-RP [SEQ. ID. NO. 23]:
TCGAGTGCGGCCGCAAGCTTGTCGACCTAATTTCTTGACTGTTTG
GCAAGCTTG.

### Embodiment 3

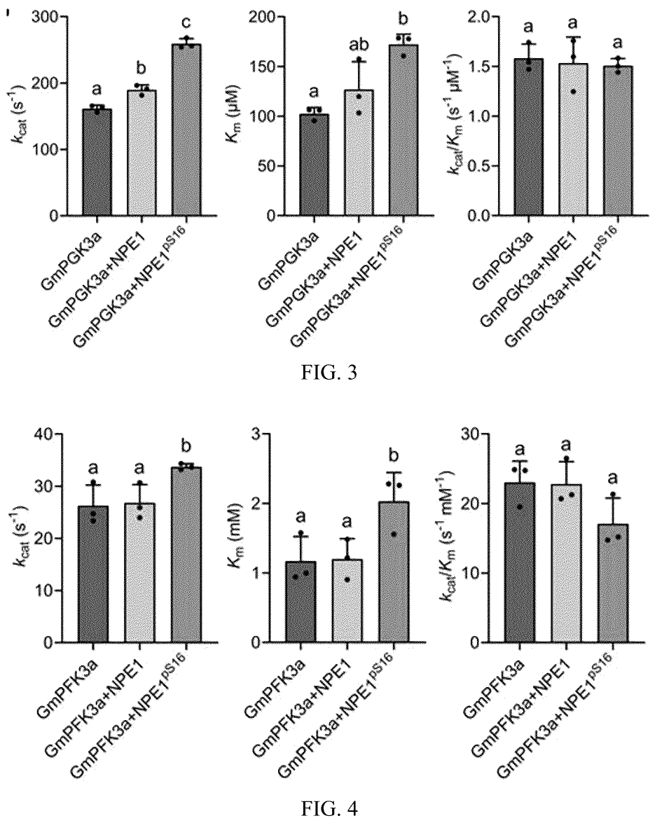

Artificially synthesize NPE1 peptide and NPE1^{pS16} peptide, a small peptide of NEP1 with phosphorylation modification of serine 16, are incubated at 4° C. for 10 minutes with His-GmPGK3a protein, expressed, purified obtained according to Embodiment 2. Initiate catalytic reaction by adding 3-phosphoglycerate (3PG) at 0.1 mM, 0.2 mM, 0.5 mM, 0.8 mM, 1 mM, 1.5 mM and 1.8 mM respectively. Then determine the reaction kinetic constants of His-GmPGK3a, His-GmPGK3a with NPE1 added, and His-GmPGK3a with NPE1^{pS16} added. It is found that the catalytic constant k_{cat} increases by approximately 17% after His-GmPGK3a binding to NPE1, and the catalytic constant k_{cat} increases by approximately 60% after binding to NPE1^{pS16}. However, NPE1 and NPE1^{pS16} also increase the catalytic Michaelis constant Kₘ of His-GmPGK3a, so the k_{cat}/Kₘ value of His-GmPGK3a is not affected, indicating that NPE1 and NPE1^{pS16} enhance the catalytic efficiency of GmPGK3a when the reaction substrate is sufficient (FIG. 3).

Artificially synthesize NPE1 and NPE1^{pS16} peptides are incubated at 4° C. for 10 minutes with His-GmPFK3a protein, which is expressed, purified and obtained according to Embodiment 2. Initiate catalytic reaction by adding fructose 6-phosphate (F6P) at 0.2 mM, 0.5 mM, 1 mM, 2.5 mM, 3.5 mM, 5 mM, 6 mM, and 8 mM respectively. Then determine the reaction kinetic constants of His-GmPFK3a, His-GmPFK3a with NPE1 added, and His-GmPFK3a with NPE1^{pS16} added. It is found that the catalytic constant k_{cat} increases by approximately 28% after His-GmPFK3a binding to NPE1^{pS16}. However, the catalytic Michaelis constant Kₘ of His-GmPFK3a is also significantly increased, so the k_{cat}/Kₘ value of His-GmPFK3a is not affected, indicating that NPE1^{pS16} enhances the catalytic efficiency of GmPFK3a when the reaction substrate is sufficient (FIG. 4).

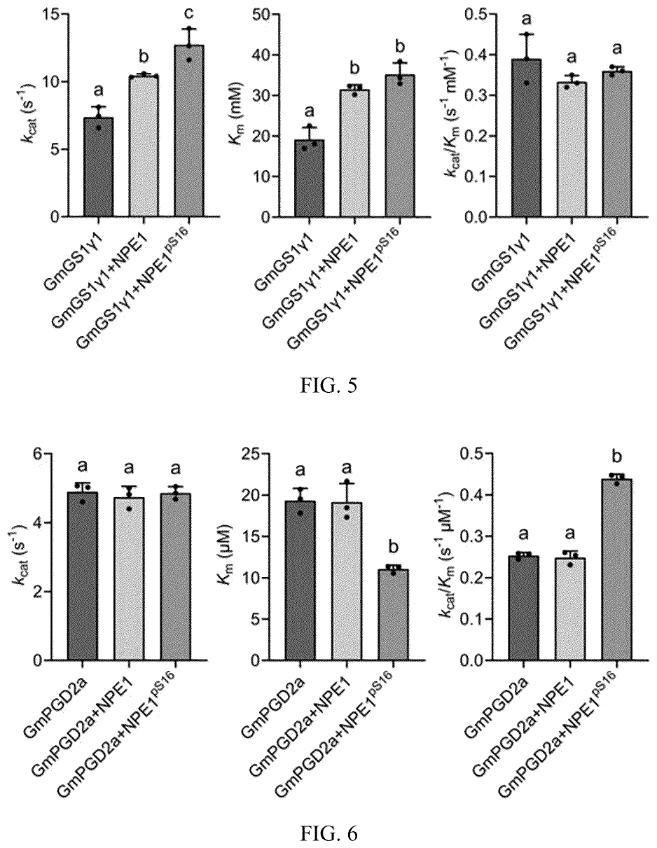

Artificially synthesize NPE1 and NPE1^{pS16} peptides are incubated at 4° C. for 10 minutes with His-GmGS1γ1 protein, which is expressed, purified and obtained according to Embodiment 2. Initiate catalytic reaction by adding glutamate (Glu) at 5 mM, 10 mM, 15 mM, 20 mM, 30 mM, 40 mM, 60 mM, 70 mM, and 80 mM respectively. Then determine the reaction kinetic constants of His-GmGS1γ1, His-GmGS1γ1 with NPE1 added, and His-GmGS1γ1 with NPE1^{pS16} added. It is found that the catalytic constant k_{cat} increases by approximately 42% after His-GmGS1γ1 binding to NPE1, and the catalytic constant k_{cat} increases by approximately 73% after binding to NPE1^{pS16}. However, NPE1 and NPE1^{pS16} also increase the catalytic Michaelis constant Kₘ of His-GmGS1γ1, so the k_{cat}/Kₘ value of His-GmGS1γ1 is not affected, indicating that NPE1 and NPE1^{pS16} enhance the catalytic efficiency of GmGS1γ1 when the reaction substrate is sufficient (FIG. 5).

Artificially synthesize NPE1 and NPE1^{pS16} peptides are incubated at 4° C. for 10 minutes with His-GmPGD2a protein, which is expressed, purified and obtained according to Embodiment 2. Initiate catalytic reaction by adding 6-phosphogluconate (6PG) at 5 μM, 10 μM, 20 μM, 50 μM, 75 μM, 100 μM and 200 μM respectively. Then determine the reaction kinetic constants of His-GmPGD2a, His-GmPGD2a with NPE1 added, and His-GmPGD2a with NPE1^{pS16} added. It is found that NPE1 and NPE1^{pS16} do not affect the catalytic constant k_{cat}. However, the catalytic Michaelis constant Kₘ of His-GmPGD2a decreases by approximately 43% after the addition of NPE1^{pS16}, and the k_{cat}/Kₘ value increases by approximately 73%, indicating that NPE1^{pS16} enhances the affinity of GmPGD2a for the 6PG substrate, thereby enhancing the catalytic efficiency of GmPGD2a when the reaction substrate is sufficient (FIG. 6).

### Embodiment 4

Using OE-NPE1-FP primer and OE-NPE1-RP primer, carry out amplification to obtain the NPE1 coding sequence (shown in SEQ ID No. 2); then insert the NPE1 coding sequence into the backbone vector pUBI3-3×FLAG-35S-mCherry to obtain pUBI3:NPE1, the NPE1 overexpression vector. Artificially synthesize coding sequence NPE1^{pS16}, where serine 16 of NPE1 is mutated to alanine, then insert the NPE1^{pS16} coding sequence into the backbone vector pUBI3-3×FLAG-35S-mCherry to obtain pUBI3:NPE1^{S16A}, the NPE1^{S16A} overexpression vector. Artificially synthesize encoding sequence NPE1^{S16D}, where serine 16 of NPE1 is mutated to aspartic acid, then insert the NPE1^{S16D} encoding sequence into the backbone vector pUBI3-3 xFLAG-35S-mCherry to obtain pUBI3:NPE1^{S16D}, the NPE1^{S16D} overexpression vector.

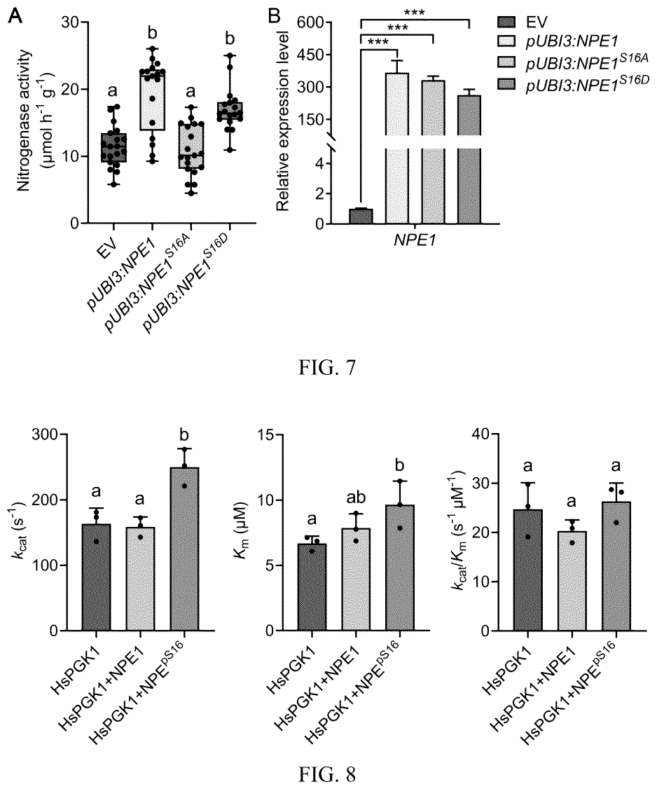

These overexpression vectors and an empty vector (EV) are transformed into Agrobacterium rhizogenes K599, and the successfully transformed K599 strain is used to infect soybean root callus. Transgenic hairy roots carrying the corresponding vectors are obtained using the hairy root transformation method. After inoculation with rhizobium USDA110, the nitrogenase activity of the transgenic hairy root nodules is measured by acetylene reduction method on the 25th day. It is found that the nitrogenase activity of pUBI3:NPE1 and pUBI3:NPE1^{S16D} transgenic root nodules increase by approximately 70% and 49%, respectively, compared with that of the empty vector transgenic root nodules, while the nitrogenase activity of pUBI3:NPE1^{S16A} transgenic root nodule is similar to that of the empty vector transgenic root nodule (FIG. 7), indicating that overexpression of the NPE1 gene significantly enhances the nitrogen fixation ability of soybean nodules, and the function of the NPE1 protein depends on the phosphorylation modification of its serine 16.

The primers are:

OE-NPE1-FP [SEQ. ID. NO. 24]:
GATTGTTGACTCGACAGTCTAGAATGATGAAATGTGAAAATATAA
TCAAAC;
OE-NPE1-RP [SEQ. ID. NO. 25]:
TGTTTGAACGATCGATGGCGCGCCTCATGATGAAACTGCAAAGTT.

### Embodiment 5

Human HsPGK1 (GeneID 5230) is a homologous gene of soybean GmPGK3a. The effect of NPE1 on the catalytic activity of human HsPGK1 protein is studied in the present invention. First, the artificially synthesized NPE1 and NPE1^{pS16} peptides are incubated with the prokaryotic expressed and purified His-HsPGK1 protein at 4° C. for 5 minutes. Then, initiate catalytic reaction by adding 3-Phosphoglycerate (3PG) at 0.01 mM, 0.02 mM, 0.05 mM, 0.1 mM, 0.2 mM and 0.5 mM respectively. Finally, determine the reaction kinetic constants of His-HsPGK1, His-HsPGK1 with NPE1 added, and His-HsPGK1 with NPE1^{pS16} added. It is found that the catalytic constant k_{cat} increases by approximately 53% after His-HsPGK1 binding to NPE1^{pS16}. However, NPE1^{pS16} also increases the catalytic Michaelis constant Kₘ of His-HsPGK1, so the k_{cat}/Kₘ value of His-HsPGK1 is not affected, indicating that NPE1^{pS16} enhances the catalytic efficiency of HsPGK1 when the reaction substrate is sufficient (FIG. 8).

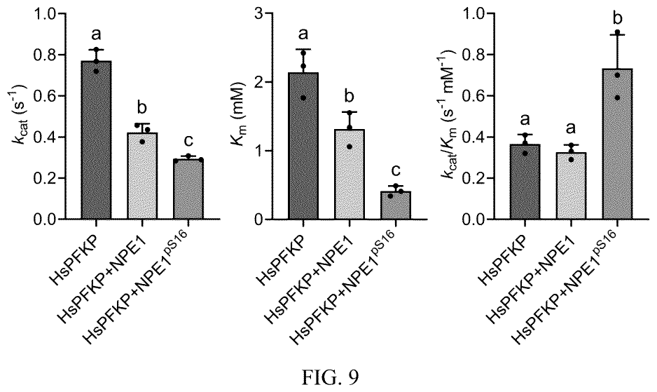

Human HsPFKP (GeneID 5214) is a homologous gene of soybean GmPFK3a. The effect of NPE1 on the catalytic activity of human HsPFKP protein is studied in the present invention. First, the artificially synthesized NPE1 and NPE1^{pS16} peptides are incubated with recombinant HsPFKP protein (MedChemExpress, HY-P75971) at 4° C. for 5 minutes. Then, initiate catalytic reaction by adding fructose 6-phosphate (F6P) at 0.5 mM, 1 mM, 2 mM, 3 mM, 4 mM, 5 mM and 7 mM respectively. Finally, determine the reaction kinetic constants of HsPFKP, HsPFKP with NPE1 added, and HsPFKP with NPE1^{pS16} added. It is found that the catalytic constant k_{cat} decreases by approximately 45% after HsPFKP binding to NPE1, while the catalytic constant k_{cat} decreases by approximately 62% after binding to NPE1^{pS16}, indicating that NPE1 and NPE1^{pS16} inhibit the catalytic efficiency of HsPFKP when the reaction substrate is sufficient (FIG. 9). However, the k_{cat}/Kₘ value increases by approximately 103% after HsPFKP binding to NPE1^{pS16}, indicating that NPE1^{pS16} significantly enhances the catalytic efficiency of HsPFKP when the reaction substrate is sufficient (FIG. 9).

The above descriptions are merely the preferred embodiments of the present invention and are not intended to be limiting. Any modifications, equivalent substitutions, improvements, etc. made within the spirit and principles of the present invention shall be included within the scope of protection of the present invention.

1. An application of overexpressed soybean NPE1 protein in enhancing catalytic efficiency of metabolic enzyme, characterized in that, said NPE1 protein comprises an amino acid sequence of SEQ ID No.3, and a serine at position 16 of said overexpressed soybean NPE1 protein is modified by phosphorylation,
said metabolic enzyme is selected from the group consisting of phosphoglycerate kinase GmPGK3a, phosphofructokinase GmPEK3a, glutamine synthetase GmGS1γ1 and 6-phosphogluconate dehydrogenase GmPGD2a, and
said metabolic enzyme is human phosphoglycerate kinase HsPGK1 or human phosphofructokinase HsPFKP.

2-5. (canceled)

6. An application of a biomaterial encoding NPE1 protein, characterized in that, said application is one or more selected from the group consisting of:
(a) improving an efficiency of symbiotic nitrogen fixation in soybean;
(b) enhancing catalytic efficiency of human phosphoglycerate kinase HsPGK1; and
(c) enhancing the catalytic efficiency of human phosphofructokinase HsPFKP,
said NPE1 protein comprises an amino acid sequence of SEQ ID No.3, and a serine at position 16 of said NPE1 protein is modified by phosphorylation,
said biomaterial is an overexpression vector containing said NPE1 protein-encoding gene, an engineered bacteria containing said NPE1 protein-encoding gene of an animal cell containing said NPE1 protein-encoding gene.

7-8. (canceled)

9. The application of a biomaterial for encoding NPE1 protein according to claim 6, characterized in that, said biomaterial has a NPE1 protein encoding gene with coding region sequence of SEQ ID No.2.

10. The application of a biomaterial for encoding NPE1 protein according to claim 6, characterized in that, said biomaterial has a nucleotide sequence of SEQ ID No.1.


SEQUENCE LISTING (27 sequences)

SEQ ID NO: 1 - DNA, 1141 bp, Glycine max
  source 1..1141: mol_type = genomic DNA
atgatgaaatgtgaaaatataatcaaacacaggatgtgggtgggttcacccaacattatgaactgtaagtatgtgtagaattatgatgtagtgcataattgataatgtcatcatgaaaaattcaattatagaaccattctttatttcttcaccatttcctagtcatttatgtgtggaaataaatttgagtgggatccaacacttttcttttatttctttttaccttgtttcatgtacctcatactctcacttttgttgctccctcctactttcacacttcctccccaatggatgcgtttgttttaagagaatagtagaaattggatagaaataagagacgaattatggattcatctgacaatgcgagatgcaagtgagagtcaatcagtaacataatattgtgcaaaattgattatatcatcatttaagattagattttagagtcgctatggttttcttaatcatttctcgccacttttgcgtgaaaatgaatttgagtgagatccaactttttttccaccctaattctatatattaaactcaagtttgctgctacttccttttttcacgtctcctcgtcaaattttttttcatctcttccccaccaaatacagtgtatacaaaaaaaataaaaaaaaaattgaacaagactgaataattgaaaacccaaaatgtccccaaacctatagtgaaacggtgcgttaagtcttctcctcctccccgaacaaaaaacactgcttataggaggactgggatcgcacttcagaggaagtcaagagtaacattaattagggcacagaacaagcagcattttgagctttgggtatttatgaacttctctctttttttactccttgtgaaaaagaattttcagttttgctatgattgacacttcaagaattttgcatgtcttggtagggcaaattctgcaagttttgtagattttttttaactaatttgatgatgtatttggctcttgctctatagaattgttagtttatatttctaatctagcttcagcagcaccatacttatatactgtaaccctcaaccttcattcctgagctaagaagttattttgaagttctgagtataattcaagtaaacaaggttttaaattgcagttgcagtttcatcatga

SEQ ID NO: 2 - DNA, 81 bp, Glycine max
  source 1..81: mol_type = genomic DNA
atgatgaaatgtgaaaatataatcaaacacaggatgtgggtgggttcacccaacattatgaactttgcagtttcatcatga

SEQ ID NO: 3 - protein, 26 aa, Glycine max
MMKCENIIKHRMWVGSPNIMNFAVSS

SEQ ID NO: 4 - DNA, 51 bp, synthetic construct
  source 1..51: mol_type = other DNA
tactatttacaattacaggtaccatgatgaaatgtgaaaatataatcaaac

SEQ ID NO: 5 - DNA, 44 bp, synthetic construct
  source 1..44: mol_type = other DNA
acgctgcccataccgccgtcgactgatgaaactgcaaagttcat

SEQ ID NO: 6 - DNA, 44 bp, synthetic construct
  source 1..44: mol_type = other DNA
caattacaggtacccggggatccatggcgactaagaggagcgtg

SEQ ID NO: 7 - DNA, 46 bp, synthetic construct
  source 1..46: mol_type = other DNA
ttgctcaccataccgccgtcgacagcatcatcaagggcaaggacac

SEQ ID NO: 8 - DNA, 49 bp, synthetic construct
  source 1..49: mol_type = other DNA
caattacaggtacccggggatccatggattctaattcttcttctccaag

SEQ ID NO: 9 - DNA, 48 bp, synthetic construct
  source 1..48: mol_type = other DNA
ttgctcaccataccgccgtcgacatgctggattgtgtcacttcccatg

SEQ ID NO: 10 - DNA, 47 bp, synthetic construct
  source 1..47: mol_type = other DNA
caattacaggtacccggggatccatgtcgttgctctccgatcttatc

SEQ ID NO: 11 - DNA, 47 bp, synthetic construct
  source 1..47: mol_type = other DNA
ttgctcaccataccgccgtcgactggtttccaaagaatggttgtctc

SEQ ID NO: 12 - DNA, 48 bp, synthetic construct
  source 1..48: mol_type = other DNA
caattacaggtacccggggatccatggctcaacccacaacaagaatag

SEQ ID NO: 13 - DNA, 48 bp, synthetic construct
  source 1..48: mol_type = other DNA
ttgctcaccataccgccgtcgacatttcttgactgtttggcaagcttg

SEQ ID NO: 14 - DNA, 52 bp, synthetic construct
  source 1..52: mol_type = other DNA
ctttaagaaggagatatactgcagatgatgaaatgtgaaaatataatcaaac

SEQ ID NO: 15 - DNA, 56 bp, synthetic construct
  source 1..56: mol_type = other DNA
gacatgctaccaccaccaccgctaccaccaccacctgatgaaactgcaaagttcat

SEQ ID NO: 16 - DNA, 49 bp, synthetic construct
  source 1..49: mol_type = other DNA
gtggacagcaaatgggtcgcggatccgcgactaagaggagcgtgggaac

SEQ ID NO: 17 - DNA, 50 bp, synthetic construct
  source 1..50: mol_type = other DNA
tcgagtgcggccgcaagcttgtcgactcaagcatcatcaagggcaaggac

SEQ ID NO: 18 - DNA, 52 bp, synthetic construct
  source 1..52: mol_type = other DNA
gtggacagcaaatgggtcgcggatccatggattctaattcttcttctccaag

SEQ ID NO: 19 - DNA, 49 bp, synthetic construct
  source 1..49: mol_type = other DNA
tcgagtgcggccgcaagcttgtcgacctagcaagttgtgggataaatgc

SEQ ID NO: 20 - DNA, 50 bp, synthetic construct
  source 1..50: mol_type = other DNA
gtggacagcaaatgggtcgcggatcctcgttgctctccgatcttatcaac

SEQ ID NO: 21 - DNA, 51 bp, synthetic construct
  source 1..51: mol_type = other DNA
tcgagtgcggccgcaagcttgtcgacttatggtttccaaagaatggttgtc

SEQ ID NO: 22 - DNA, 51 bp, synthetic construct
  source 1..51: mol_type = other DNA
gtggacagcaaatgggtcgcggatccatggctcaacccacaacaagaatag

SEQ ID NO: 23 - DNA, 54 bp, synthetic construct
  source 1..54: mol_type = other DNA
tcgagtgcggccgcaagcttgtcgacctaatttcttgactgtttggcaagcttg

SEQ ID NO: 24 - DNA, 51 bp, synthetic construct
  source 1..51: mol_type = other DNA
gattgttgactcgacagtctagaatgatgaaatgtgaaaatataatcaaac

SEQ ID NO: 25 - DNA, 45 bp, synthetic construct
  source 1..45: mol_type = other DNA
tgtttgaacgatcgatggcgcgcctcatgatgaaactgcaaagtt

SEQ ID NO: 26 - DNA, 20 bp, synthetic construct
  source 1..20: mol_type = other DNA
atgatgaaatgtgaaaatat

SEQ ID NO: 27 - DNA, 20 bp, synthetic construct
  source 1..20: mol_type = other DNA
tcatgatgaaactgcaactg